(19) United States
(12) Patent Application Publication
(10) Pub. No.: US 2007/0054929 A1
(43) Pub. Date: Mar. 8, 2007

(54) 2-Substituted pyrimidines

(75) Inventors: Jordi Tormo i Blasco, Laudenbach (DE); Carsten Blettner, Hong Kong (CN); Bernd Muller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Siegfried Strathmann, Limburgerhof (DE); Ulrich Schofl, Bruhl (DE); Maria Scherer, Godramstein (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF AKTIENGESELLSCHAFT, 67056 LUDWIGSHAFEN (DE)

(21) Appl. No.: 10/555,894

(22) PCT Filed: May 10, 2004
(86) PCT No.: PCT/EP04/04957

§ 371 Date: Nov. 7, 2005

(30) Foreign Application Priority Data
May 20, 2003 (DE) 103 23 026.2

(51) Int. Cl. A61K 31/506; C07D 403/04

(52) U.S. Cl. 514/275; 544/329

(57) ABSTRACT

The present invention relates to pyrimidines of the formula I 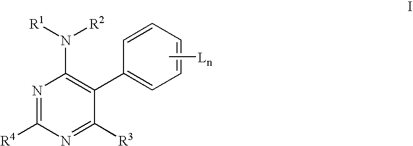
in which the index n and the substituents L, R¹-R³ are as defined in the description and
R⁴ corresponds to one of the formulae 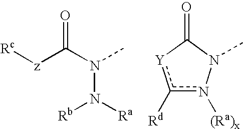 and to processes and intermediates for preparing these compounds, to compositions comprising them and to their use for controlling phytopathogenic harmful fungi.

The invention relates to 2-substituted pyrimidines of the formula I

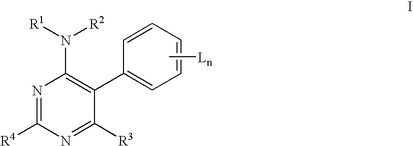

in which the index and the substituents are as defined below:

n is an integer from 1 to 5, where at least one substituent L is located in the ortho-position on the phenyl ring;

L is halogen, cyano, cyanato (OCN), nitro, C₁-C₈-alkyl, C₂-C₁₀-alkenyl, C₂-C₁₀-alkynyl, C₁-C₆-alkoxy, —C(═O)—A, —C(═O)—O—A, —C(═O)—N(A′)A, C(A′)(═N—OA), N(A′)A, N(A′)—C(═O)—A, N(A″)—C(═O)—N(A′)A, S(═O)ₘ—A, S(═O)ₘ—O—A or S(═O)ₘ—N(A′)A,
m is 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by cyano or C₁-C₄-alkoxy; or A and A′ together with the atoms to which they are attached are a five- or six-membered saturated, partially unsaturated or aromatic heterocycle which contains one to four heteroatoms from the group consisting of O, N and S;
where the aliphatic, alicyclic or aromatic groups of the radical definitions of L for their part may be partially or fully halogenated or may carry one to four groups R^{u}:
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)—A, —C(′O)—O—A, —C(═O)—N(A′)A, C(A′)(═N—OA), N(A′)A, N(A′)—C(═O)—A, N(A″)—C(═O)—N(A′)A, S(═O)ₘ—A, S(═O)ₘ—O—A or S(═O)ₘ—N(A′)A, where m, A, A′, A″ are as defined above and where the aliphatic, alicyclic or aromatic groups for their part may be partially or fully halogenated or may carry one to three groups R^{v}, R^{v} having the same meaning as R^{u};
R¹, R² independently of one another are C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl or C₂-C₆-haloalkynyl;
   R² may additionally be hydrogen;
R¹ and R² may also, together with the nitrogen atom to which they are attached, form a saturated or unsaturated five- or six-membered ring which may be interrupted by an ether (—O—), carbonyl C[═O])-, thio (—S—), sulfoxyl (—S[═O]—) or sulfenyl (—SO₂—) group;
R³ is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy, where the alkyl, alkenyl and alkynyl radicals of R³ may be substituted by halogen, cyano, nitro, C₁-C₂-alkoxy or C₁-C₄-alkoxycarbonyl;
R⁴ corresponds to one of the formulae 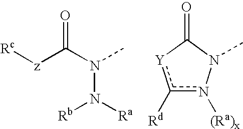
   where
x is 0 or 1;
R^{a}, R^{b} and R^{c′} independently of one another are hydrogen, C₁-C₆-alkyl, C₂-C₈-alkenyl, C₂-C₈-alkynyl, C₃-C₆-cycloalkyl, C₄-C₆-cycloalkenyl,
R^{a}, R^{b} together with the nitrogen atom to which they are attached may have the meaning R^{c}—Z—C(R^{d})═N;
Z is oxygen or N—R^{c};
Y is C(H)—R^{e}, C—R^{e}, N—N(H)—R^{c} or N—R^{c};
[symbol] may be a double bond or a single bond;
R^{d}, R^{e} have the same meanings as R^{c} and may additionally be halogen or cyano;
R^{d} together with the carbon to which it is attached may be a carbonyl group;
   where the aliphatic, alicyclic or aromatic groups of the radical definitions of R^{a}, R^{b}, R^{c}, R^{d} or R^{e} for their part may be partially or fully halogenated or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C6-cycloalkenyloxy, and where two of the radicals R^{a}, R^{b} or R^{c} together with the atoms to which they are attached may form a five- or six-membered saturated, partially unsaturated or aromatic heterocycle which contains one to four heteroatoms from the group consisting of O, N and S.

Moreover, the invention relates to a process for preparing these compounds, to compositions comprising them and to their use for controlling phytopathogenic harmful fungi.

Fungicidal pyrimidines carrying a cyanamino substituent in the 2-position are known from WO-A 01/96314. Furthermore, fungicidal pyrimidines carrying generally a heterocyclyl radical in the 2-position are known from WO 02/74753. However, the only pyrimidines specifically disclosed are heteroaryl-substituted pyrimidines.

However, in many cases the activity of the abovementioned pyrimidines is unsatisfactory. It was an object of the present invention to provide compounds having improved activity.

We have found that this object is achieved by the pyrimidines of the formula I defined at the outset. Moreover, we have found processes for their preparation and compositions comprising them for controlling harmful fungi.

The compounds I can be obtained by different routes.
1) It is possible, for example, to use the hydrazinopyrimidines of the formula 11, whose preparation is described in detail in WO-A 02/074753 or DE 10156279.9, as starting materials. A preferred preparation of the compounds II starting from sulfones IIa is shown below.

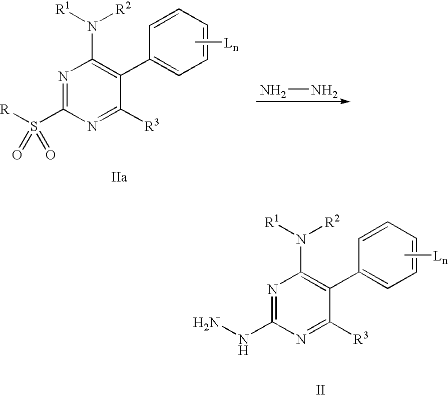

The further synthesis can be carried out as shown in Scheme 1:

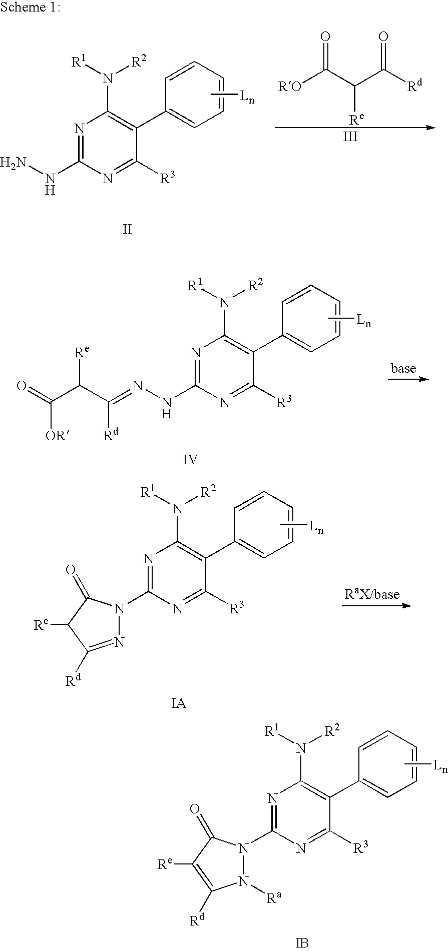

The hydrazine compound II is condensed with a dicarbonyl compound III where the substituents R¹, R², R³, Lₙ, R^{d} and R^{e} are as defined above and R′ is an alkyl, aryl or benzyl group (see Scheme 1), giving the compounds of the formula VI. The dicarbonyl compounds of the formula III are known from Angew. Chem. Int. Ed. Engl. 1989 28, p. 500. The condensation is carried out as described in detail in DE 19627002. Cyclization to the compounds IA according to the invention is carried out, for example, in the presence of bases, such as, in particular, alkali metal alkoxides. The reaction with sodium methoxide is described explicitly (Synlett 1996, 667-8). In the presence of alkylating agent R^{a}X, where R^{a} is as defined above and X is a leaving group, such as halide or sulfate, and a strong base, such as, for example, sodium hydride or anhydrous potassium carbonate, the compounds IB according to the invention are obtained.
2) The preparation process shown in Scheme 2 affords the compounds IC according to the invention.

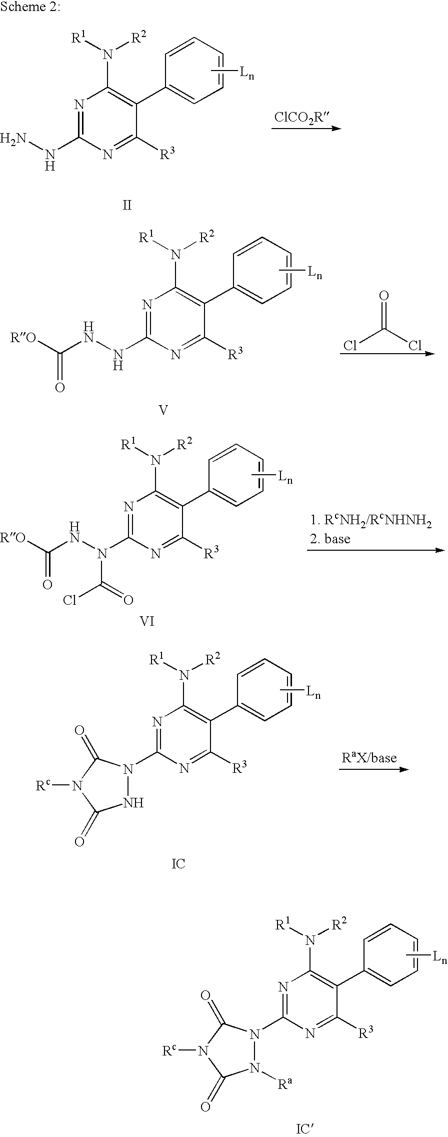

The synthesis of the compounds IC and IC′ preferably starts with the hydrazine compound II, the preparation of which has already been described in detail above. The reaction with chloroformic esters (R″ is an alkyl radical), giving the acylated compounds V, is generally carried out in the presence of a base. Further reaction of V with phosgene or a phosgene equivalent, giving VI, and subsequent cyclization in the presence of an amine/hydrazine and a base can be carried out analogously to the method described in Chem. Ber. 1898, 31, page 2320 ff. Cyclization in the presence of amines R^{c}NH₂ gives triazolidinediones IC, whereas cyclization in the presence of hydrazines R^{c}NH—NH₂ gives the compounds IC″.

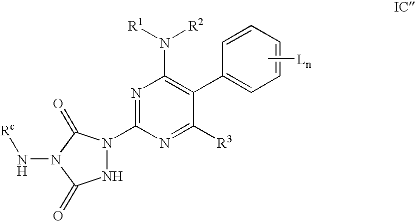

The alkylation of the compounds IC with alkylating agent R^{a}X, where R^{a} is as defined above and X is a leaving group, such as halide or sulfate, in the presence of a base is carried out according to DE 3336693.
3) Triazolidinones of type ID can advantageously be synthesized as shown in Scheme 3.

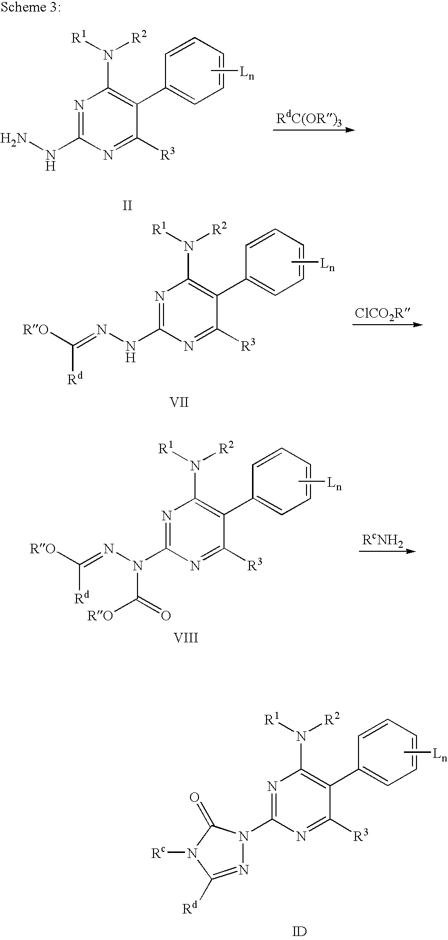

Starting with the hydrazine compound II and orthoesters, the condensed compound VII is obtained analogously to the method described in J. Am. Chem. Soc. 1995, 77, p.1148. VII is further acylated with chloroformic esters to give VIII, analogously to the method described in Compt. Rend. Acad. Sci. 1981, 293, N8, 573-76. R″ in the orthoester and in the chloroformic ester is C₁-C₆-alkyl. Cyclization to the compounds ID according to the invention is carried out in the presence of amines R^{c}NH₂. If, instead of amines, hydrazines of the formula R^{c}NH—NH₂ are used, triazolidinones of the formula ID″ are obtained.

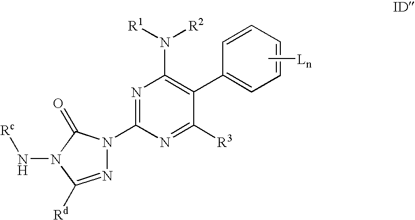

The radical R³ (in particular alkyl) in the 6-position or in the pyrimidine ring can be introduced by reaction with transition metal catalysis, such as Ni or Pd catalysis. In some cases, it may be advisable to change the order and to introduce substituent R³ prior to substituent NR¹R².

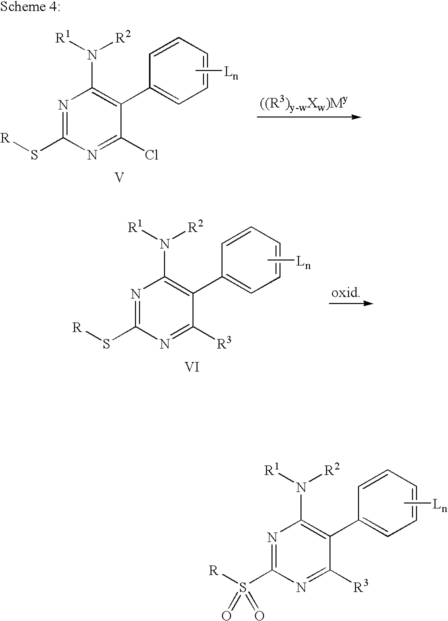

In the formula (R³)_{y-w}X_{w}-M^{y}, M is a metal ion of valency Y, such as, for example, B, Zn, Mg, Cu or Sn, X is chlorine, bromine, iodine or hydroxyl, R³ is preferably C₁-C₄-alkyl and w is a number from 0 to 3. This reaction can be carried out, for example, analogously to the following methods: J. Chem. Soc. Perkin Trans. 1, 1187 (1994), ibit. 1, 2345 (1996); WO-A 99/41255; Aust. J. Chem., Vol. 43, 733 (1990); J. Org. Chem., Vol. 43, 358 (1978); J. Chem. Soc. Chem. Commun. 866 (1979); Tetrahedron Lett., Vol. 34, 8267 (1993); ibit., Vol. 33, 413 (1992).

What was said above refers in particular to the preparation of compounds in which R³ is an alkyl group. If R³ is a cyano group or an alkoxy substituent, the radical R³ can be introduced by reaction with alkali metal cyanides and alkali metal alkoxides, respectively.

In the definitions of the symbols given in the formulae above, collective terms were used which are generally representative for the following substituents:

halogen: fluorine, chlorine, bromine and iodine;

alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 10 carbon atoms (as mentioned above), where in these groups some or all of the hydrogen atoms may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6, 8 or 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkadienyl: unsaturated straight-chain or branched hydrocarbon radicals having 4, 6, 8 or 10 carbon atoms and two double bonds in any position;

haloalkenyl: unsaturated straight-chain or branched hydrocarbon radicals having 2 to 10 carbon atoms and a double bond in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6, 8 or 10 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- or six-membered saturated, partially unsaturated or aromatic heterocycle which contains one to four heteroatoms from the group consisting of O, N and S:
5- or 6-membered heterocyclyl which contains one to three nitrogen atoms and/or one oxygen or sulfur atom or one or two oxygen and/or sulfur atoms, for example 2-tetrahydrofuryl, 3-tetrahydrofuryl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2, 3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which contains one to three or one to four nitrogen atoms: 6-membered heteroaryl groups which, in addition to carbon atoms, may contain one to three and one to four nitrogen atoms, respectively, as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The scope of the present invention includes the (R) and (S) isomers and the racemates (±) of compounds of the formula I having chiral centers.

Hereinbelow, the embodiments of the invention are described in more detail.

With a view to the intended use of the pyrimidines of the formula I, particular preference is given to the following meanings of the substituents, in each case on their own or in combination:

Preference is given to compounds I in which R¹ is C₁-C₆-alkyl, C₁-C₆-haloalkyl, C₂-C₆-alkenyl, C₂-C₆-alkynyl or C₃-C₆-cycloalkyl and R² is hydrogen.

Especially preferred are compounds I in which R¹ is C₁-C₆-haloalkyl or C₁-C₆-alkyl branched in the α-position.

In addition, preference is given to compounds I in which R¹ is C₁-C₄-haloalkyl and R² is hydrogen.

Moreover, preference is given to compounds I in which R¹ and R² together with the nitrogen to which they are attached form a five- or six-membered ring which may be interrupted by an oxygen atom and may carry one or two C₁-C₆-alkyl substituents.

Especially preferred are groups NR¹R² such as—in particular in the α-position—methylated pyrrolidines or piperidines.

Moreover, particular preference is given to pyrimidines I in which the index n and the substituents L¹ to L⁵ are as defined below:

n is 1 to 3;

L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, —C(═O)—O—A, —C(═O)—N(A′)A, C(A′)(═N—OA), N(A′)A, N(A′)—C(═O)—A or S(═O)ₘ—A;
m is 0, 1 or 2;
A, A′, A″ independently of one another are hydrogen, C₁-C₆-alkyl, C₂-C₆-alkenyl, C₂-C₆-alkynyl, where the organic radicals may be partially or fully halogenated or may be substituted by cyano or C₁-C₄-alkoxy, or A and A′ together with the atoms to which they are attached are a five- or six-membered saturated heterocycle which contains one to four heteroatoms from the group consisting of O, N and S.

Especially preferred are pyrimidines I where the substituents L¹ to L5 are as defined below:

L is halogen, cyano, C₁-C₈-alkyl, C₁-C₆-alkoxy, —C(═O)—O—A, —C(═O)—N(A′)A,
m is 0, 1 or 2;
A, A′, A″ independently of one another are hydrogen, C₁-C₆-alkyl, C₂-C₆-alkenyl, C₂-C₆-alkynyl.

Particular preference is given to compounds I in which R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₅-C₆-cycloalkenyl, —C(═O)—O—A, —C(═O)—N(A′)A, C(A′)(═N—OA), where the aliphatic or alicyclic groups for their part may be partially or fully halogenated or may carry one to three groups R^{v}, R^{v} having the same meaning as R^{u}.

Especially preferred are compounds I in which R^{u} is halogen, cyano, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₃-C₆-cycloalkyl, C₅-C₆-cycloalkenyl.

Moreover, preference is given to pyrimidines I where the phenyl group substituted by Lₙ is the group B 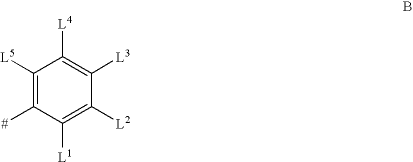
where # is the point of attachment to the pyrimidine skeleton and
L¹ is fluorine, chlorine, CH₃ or CF₃;
L²,L⁴ independently of one another are hydrogen, CH₃ or fluorine;
L³ is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L⁵ is hydrogen, fluorine, chlorine or CH₃.

Particular preference is also given to compounds I in which R³ is C₁-C₄-alkyl which may be substituted by halogen.

Moreover, particular preference is given to compounds I in which R³ is halogen, cyano, C₁-C₄-alkyl or C₁-C₄-alkoxy.

Especially preferred are compounds I in which R³ is methyl, ethyl, cyano, bromine or in particular chlorine.

Furthermore, preference is given to pyrimidines of the formula I in which R⁴ is

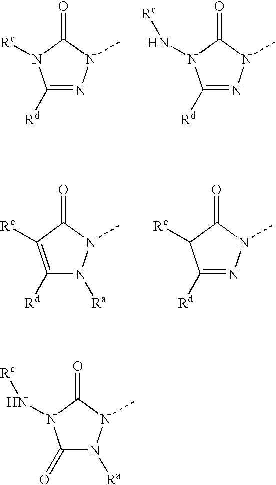

Especially preferred are pyrimidines of the formula I in which R⁴ is.

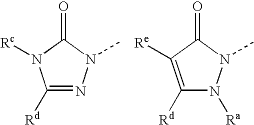

Especially preferred are pyrimidines of the formula I in which R⁴ is

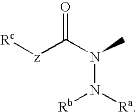

R^{a}, R^{b} and R^{c} are preferably independently of one another hydrogen, C₁-C₆-alkyl, C₂-C₆-alkenyl, C₂-C₆-alkynyl or C₃-C₆-cycloalkyl.

Preferably, R^{a}, R^{b} and R^{c,} independently of one another, are hydrogen, methyl or ethyl.

For the intermediates of the formulae IV, V, VI and VII, the same preferences as mentioned above for the active compounds apply. Here, the preferred meanings of the substituents apply in each case on their own and in combination with other preferences.

Especially preferred are intermediates of the formulae IV and V.

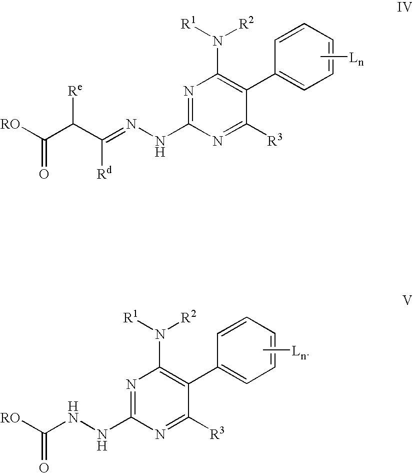

In particular with a view to their use, preference is given to compounds I compiled in the tables below. Moreover, the groups mentioned for a substituent in the tables are per se, independently of the combination in which they are mentioned, a particularly preferred embodiment of the substituent in question.

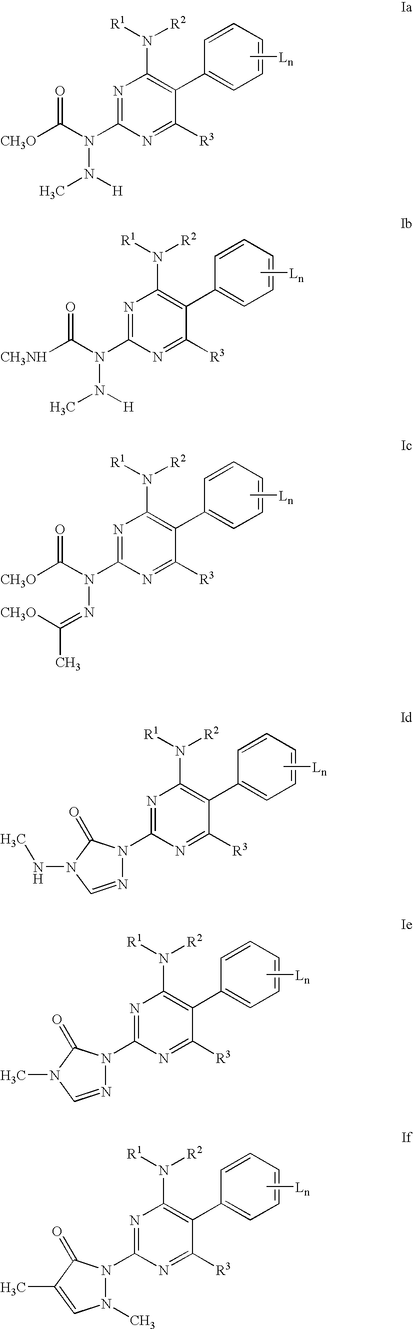

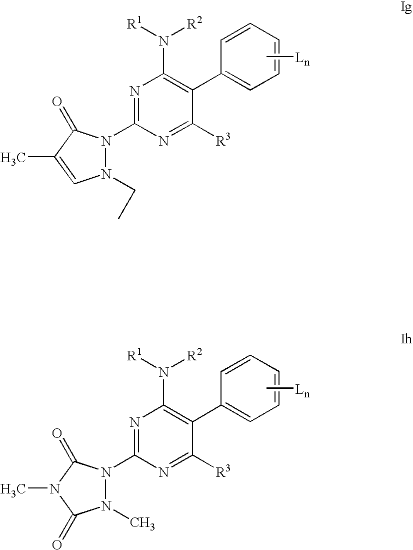

Compounds of the formulae Ia, Ib, Ic, Id, Ie, If, Ig and Ih in which Lₙ is 2-fluoro,6-chloro, R³ is methyl and R¹, R²for each compound corresponds to one row of Table A

Table 1

Compounds of the formulae Ia, Ib, Ic, Id, Ie, If, Ig and Ih in which Lₙ is 2,6-difluoro, R³ is methyl and R¹, R² for each compound corresponds to one row of Table A

Table 2

Compounds of the formulae Ia, Ib, Ic, Id, Ie, If, Ig and Ih in which Lₙ is 2,6-dichloro, R³ is methyl and R¹, R² for each compound corresponds to one row of Table A

Table 3

Compounds of the formulae Ia, Ib, Ic, Id, Ie, If, Ig and Ih in which Lₙ is 2-fluoro,6-methyl, R³ is methyl and R¹, R² for each compound corresponds to one row of Table A

Table 4

Compounds of the formulae Ia, Ib, Ic, Id, Ie, If, Ig and Ih in which Lₙ is 2,4,6-trifluoro, R³ is methyl and R¹, R² for each compound corresponds to one row of Table A

Table 5

Compounds of the formulae Ia, Ib, Ic, Id, Ie, If, Ig and Ih in which Lₙ is 2-methyl,4-fluoro, R³is methyl and R¹, R² for each compound corresponds to one row of Table A

Table 6

Compounds of the formulae Ia, Ib, Ic, Id, Ie, If, Ig and Ih in which Lₙ is 2-fluoro,4-methoxycarbonyl, R³is methyl and R¹, R² for each compound corresponds to one row of Table A

Table 7

Compounds of the formulae Ia, Ib, Ic, Id, Ie, If, Ig and Ih in which Lₙ is 2-fluoro,4-CN, R³ is methyl and R¹, R² for each compound corresponds to one row of Table A

Table 8

Compounds of the formulae Ia, Ib, Ic, Id, Ie, If, Ig and Ih in which Lₙ is 2,4,5-trifluoro, R³ is methyl and R¹, R² for each compound corresponds to one row of Table A

Table 9

Compounds of the formulae Ia, Ib, Ic, Id, Ie, If, Ig and Ih in which Lₙ is 2,4-dichloro, R³ is methyl and R¹, R² for each compound corresponds to one row of Table A

Table 10

Compounds of the formulae Ia, Ib, Ic, Id, Ie, If, Ig and Ih in which Lₙ is 2-chloro, R³ is methyl and R¹, R² for each compound corresponds to one row of Table A

Table 11

Compounds of the formulae Ia, Ib, Ic, Id, Ie, If, Ig and Ih in which Lₙ is 2-fluoro, R³ is methyl and R¹, R² for each compound corresponds to one row of Table A

Table 12

Compounds of the formulae Ia, Ib, Ic, Id, Ie, If, Ig and Ih in which Lₙ is 2,4-difluoro, R³ is methyl and R¹, R² for each compound corresponds to one row of Table A

Table 13

Compounds of the formulae Ia, Ib, Ic, Id, Ie, If, Ig and Ih in which Lₙ is 2-fluoro-4-chloro, R³is methyl and R¹, R² for each compound corresponds to one row of Table A

Table 14

Compounds of the formulae Ia, Ib, Ic, Id, Ie, If, Ig and Ih in which Lₙ is 2-chloro-4-fluoro, R³is methyl and R¹, R² for each compound corresponds to one row of Table A

Table 15

Compounds of the formulae Ia, Ib, Ic, Id, Ie, If, Ig and Ih in which Lₙ is 2,3-difluoro, R³ is methyl and R¹, R² for each compound corresponds to one row of Table A

Table 16

Compounds of the formulae Ia, Ib, Ic, Id, Ie, If, Ig and Ih in which Lₙ is 2,5-difluoro, R³ is methyl and R¹, R² for each compound corresponds to one row of Table A

Table 17

Compounds of the formulae Ia, Ib, Ic, Id, Ie, If, Ig and Ih in which Lₙ is 2,3,4-trifluoro, R³ is methyl and R¹, R² for each compound corresponds to one row of Table A

Table 18

Compounds of the formulae Ia, Ib, Ic, Id, Ie, If, Ig and Ih in which Lₙ is 2-methyl, R³ is methyl and R¹, R² for each compound corresponds to one row of Table A

Table 19

Compounds of the formulae Ia, Ib, Ic, Id, Ie, If, Ig and Ih in which Lₙ is 2,4-dimethyl, R³ is methyl and R¹, R² for each compound corresponds to one row of Table A

Table 20

Compounds of the formulae Ia, Ib, Ic, Id, Ie, If, Ig and Ih in which Lₙ is 2-methyl-4-chloro, R³ is methyl and R¹, R² for each compound corresponds to one row of Table A

Table 21

Compounds of the formulae Ia, Ib, Ic, Id, Ie, If, Ig and Ih in which Lₙ is 2-fluoro-4-methyl, R³ is methyl and R¹, R² for each compound corresponds to one row of Table A

Table 22

Compounds of the formulae Ia, Ib, Ic, Id, Ie, If, Ig and Ih in which Lₙ is 2,6-dimethyl, R³ is methyl and R¹, R² for each compound corresponds to one row of Table A

Table 23

Compounds of the formulae Ia, Ib, Ic, Id, Ie, If, Ig and Ih in which Lₙ is 2,4,6-trimethyl, R³ is methyl and R¹, R² for each compound corresponds to one row of Table A

Table 24

Compounds of the formulae Ia, Ib, Ic, Id, Ie, If, Ig and Ih in which Lₙ is 2,6-difluoro-4-cyano, R³ is methyl and R¹, R² for each compound corresponds to one row of Table A

Table 25

Compounds of the formulae Ia, Ib, Ic, Id, Ie, If, Ig and Ih in which Lₙ is 2,6-difluoro-4-methyl, R³ is methyl and R¹, R² for each compound corresponds to one row of Table A

Table 26

Compounds of the formulae Ia, Ib, Ic, Id, Ie, If, Ig and Ih in which Lₙ is 2,6-difluoro-4-methoxycarbonyl, R³ is methyl and R¹, R² for each compound corresponds to one row of Table A

Table 27

Compounds of the formulae Ia, Ib, Ic, Id, Ie, If, Ig and Ih in which Lₙ is 2-chloro,4-methoxy, R³ is methyl and R¹, R² for each compound corresponds to one row of Table A

Table 28

Compounds of the formulae Ia, Ib, Ic, Id, Ie, If, Ig and Ih in which Lₙ is 2-chloro,4-methyl, R³ is methyl and R¹, R² for each compound corresponds to one row of Table A

Table 29

Compounds of the formulae Ia, Ib, Ic, Id, Ie, If, Ig and Ih in which Lₙ is 2-chloro,4-methoxycarbonyl, R³ is methyl and R¹, R² for each compound corresponds to one row of Table A

Table 30

Compounds of the formulae Ia, Ib, Ic, Id, Ie, If, Ig and Ih in which Lₙ is 2-chloro,4-bromo, R³is methyl and R¹, R² for each compound corresponds to one row of Table A

Table 31

Compounds of the formulae Ia, Ib, Ic, Id, Ie, If, Ig and Ih in which Lₙ is 2-chloro,4-cyano, R³ is methyl and R¹, R² for each compound corresponds to one row of Table A

Table 32

Compounds of the formulae Ia, Ib, Ic, Id, Ie, If, Ig and Ih in which Lₙ is 2,6-difluoro,4-methoxy, R³ is methyl and R¹, R² for each compound corresponds to one row of Table A

Table 33

Compounds of the formulae Ia, Ib, Ic, Id, Ie, If, Ig and Ih in which Lₙ is 2-fluoro,3-methyl, R³is methyl and R¹, R² for each compound corresponds to one row of Table A

Table 34

Compounds of the formulae Ia, Ib, Ic, Id, Ie, If, Ig and Ih in which Lₙ is 2,5-dimethyl, R³ is methyl and R¹, R² for each compound corresponds to one row of Table A

Table 35

Compounds of the formulae Ia, Ib, Ic, Id, Ie, If, Ig and Ih in which Lₙ is 2-methyl,4-cyano, R³is methyl and R¹, R² for each compound corresponds to one row of Table A

Table 36

Compounds of the formulae Ia, Ib, Ic, Id, Ie, If, Ig and Ih in which Lₙ is 2-methyl,4-bromo, R³is methyl and R¹, R² for each compound corresponds to one row of Table A

Table 37

Compounds of the formulae Ia, Ib, Ic, Id, Ie, If, Ig and Ih in which Lₙ is 2-methyl,5-fluoro, R³is methyl and R¹, R² for each compound corresponds to one row of Table A

Table 38

Compounds of the formulae Ia, Ib, Ic, Id, Ie, If, Ig and Ih in which Lₙ is 2-methyl,4-methoxy, R³ is methyl and R¹, R² for each compound corresponds to one row of Table A

Table 39

Compounds of the formulae Ia, Ib, Ic, Id, Ie, If, Ig and Ih in which Lₙ is 2-methyl,4-methoxycarbonyl, R³is methyl and R¹, R² for each compound corresponds to one row of Table A

Table 40

Compounds of the formulae Ia, Ib, Ic, Id, Ie, If, Ig and Ih in which Lₙ is 2,5-dimethyl,4-bromo, R³is methyl and R¹, R² for each compound corresponds to one row of Table A

Table 41

Compounds of the formulae Ia, Ib, Ic, Id, Ie, If, Ig and Ih in which Lₙ is 2-fluoro,4-bromo, R³is methyl and R¹, R² for each compound corresponds to one row of Table A

Table 42

Compounds of the formulae Ia, Ib, Ic, Id, Ie, If, Ig and Ih in which Lₙ is 2-fluoro,4-methoxy, R³ is methyl and R¹, R² for each compound corresponds to one row of Table A

Table 43

Compounds of the formulae Ia, Ib, Ic, Id, Ie, If, Ig and Ih in which Lₙ is 2-fluoro,5-methyl, R³ is methyl and R¹, R² for each compound corresponds to one row of Table A

Table 44

Compounds of the formulae Ia, Ib, Ic, Id, Ie, If, Ig and Ih in which Lₙ is pentafluoro, R³ is methyl and R¹, R² for each compound corresponds to one row of Table A

Table 45

Compounds of the formulae Ia, Ib, Ic, Id, Ie, If, Ig and Ih in which Lₙ is 2-fluor,6-chloro, R³ is chlorine and R¹, R² for each compound corresponds to one row of Table A

Table 46

Compounds of the formulae Ia, Ib, Ic, Id, Ie, If, Ig and Ih in which Lₙ is 2,6-difluoro, R³ is chlorine and R¹, R² for each compound corresponds to one row of Table A

Table 47

Compounds of the formulae Ia, Ib, Ic, Id, Ie, If, Ig and Ih in which Lₙ is 2,6-dichloro, R³ is chlorine and R¹, R² for each compound corresponds to one row of Table A

Table 48

Compounds of the formulae Ia, Ib, Ic, Id, Ie, If, Ig and Ih in which Lₙ is 2-fluoro,6-methyl, R³is chlorine and R¹, R² for each compound corresponds to one row of Table A

Table 49

Compounds of the formulae Ia, Ib, Ic, Id, Ie, If, Ig and Ih in which Lₙ is 2,4,6-trifluoro, R³ is chlorine and R¹, R² for each compound corresponds to one row of Table A

Table 50

Compounds of the formulae Ia, Ib, Ic, Id, Ie, If, Ig and Ih in which Lₙ is 2-methyl,4-fluoro, R³is chlorine and R¹, R² for each compound corresponds to one row of Table A

Table 51

Compounds of the formulae Ia, Ib, Ic, Id, Ie, If, Ig and Ih in which Lₙ is 2-fluoro,4-methoxycarbonyl, R³is chlorine and R¹, R² for each compound corresponds to one row of Table A

Table 52

Compounds of the formulae Ia, Ib, Ic, Id, Ie, If, Ig and Ih in which Lₙ is 2-fluoro,4-CN, R³ is chlorine and R¹, R² for each compound corresponds to one row of Table A

Table 53

Compounds of the formulae Ia, Ib, Ic, Id, Ie, If, Ig and Ih in which Lₙ is 2,4,5-trifluoro, R³ is chlorine and R¹, R² for each compound corresponds to one row of Table A

Table 54

Compounds of the formulae Ia, Ib, Ic, Id, Ie, If, Ig and Ih in which Lₙ is 2,4-dichloro, R³ is chlorine and R¹, R² for each compound corresponds to one row of Table A

Table 55

Compounds of the formulae Ia, Ib, Ic, Id, Ie, If, Ig and Ih in which Lₙ is 2-chloro, R³is chlorine and R¹, R² for each compound corresponds to one row of Table A

Table 56

Compounds of the formulae Ia, Ib, Ic, Id, Ie, If, Ig and Ih in which Lₙ is 2-fluoro, R³ is chlorine and R¹, R² for each compound corresponds to one row of Table A

Table 57

Compounds of the formulae Ia, Ib, Ic, Id, Ie, If, Ig and Ih in which Lₙ is 2,4-difluoro, R³ is chlorine and R¹, R² for each compound corresponds to one row of Table A

Table 58

Compounds of the formulae Ia, Ib, Ic, Id, Ie, If, Ig and Ih in which Lₙ is 2-fluoro-4-chloro, R³ is chlorine and R¹, R² for each compound corresponds to one row of Table A

Table 59

Compounds of the formulae Ia, Ib, Ic, Id, Ie, If, Ig and Ih in which Lₙ is 2-chloro-4-fluoro, R³ is chlorine and R¹, R² for each compound corresponds to one row of Table A

Table 60

Compounds of the formulae Ia, Ib, Ic, Id, Ie, If, Ig and Ih in which Lₙ is 2,3-difluoro, R³is chlorine and R¹, R² for each compound corresponds to one row of Table A

Table 61

Compounds of the formulae Ia, Ib, Ic, Id, Ie, If, Ig and Ih in which Lₙ is 2,5-difluoro, R³ is chlorine and R¹, R² for each compound corresponds to one row of Table A

Table 62

Compounds of the formulae Ia, Ib, Ic, Id, Ie, If, Ig and Ih in which Lₙ is 2,3,4-trifluoro, R³ is chlorine and R¹, R² for each compound corresponds to one row of Table A

Table 63

Compounds of the formulae Ia, Ib, Ic, Id, Ie, If, Ig and Ih in which Lₙ is 2-methyl, R³ is chlorine and R¹, R² for each compound corresponds to one row of Table A

Table 64

Compounds of the formulae Ia, Ib, Ic, Id, Ie, If, Ig and Ih in which Lₙ is 2,4-dimethyl, R³ is chlorine and R¹, R² for each compound corresponds to one row of Table A

Table 65

Compounds of the formulae Ia, Ib, Ic, Id, Ie, If, Ig and Ih in which Lₙ is 2-methyl-4-chloro, R³ is chlorine and R¹, R² for each compound corresponds to one row of Table A

Table 66

Compounds of the formulae Ia, Ib, Ic, Id, Ie, If, Ig and Ih in which Lₙ is 2-fluoro-4-methyl, R² is chlorine and R¹, R² for each compound corresponds to one row of Table A

Table 67

Compounds of the formulae Ia, Ib, Ic, Id, Ie, If, Ig and Ih in which Lₙ is 2,6-dimethyl, R³ is chlorine and R¹, R² for each compound corresponds to one row of Table A

Table 68

Compounds of the formulae Ia, Ib, Ic, Id, Ie, If, Ig and Ih in which Lₙ is 2,4,6-trimethyl, R³ is chlorine and R¹, R² for each compound corresponds to one row of Table A

Table 69

Compounds of the formulae Ia, Ib, Ic, Id, Ie, If, Ig and Ih in which Lₙ is 2,6-difluoro-4-cyano, R³ is chlorine and R¹, R² for each compound corresponds to one row of Table A

Table 70

Compounds of the formulae Ia, Ib, Ic, Id, Ie, If, Ig and Ih in which Lₙ is 2,6-difluoro-4-methyl, R³ is chlorine and R¹, R² for each compound corresponds to one row of Table A

Table 71

Compounds of the formulae Ia, Ib, Ic, Id, Ie, If, Ig and Ih in which Lₙ is 2,6-difluoro-4-methoxycarbonyl, R³ is chlorine and R¹, R² for each compound corresponds to one row of Table A

Table 72

Compounds of the formulae Ia, Ib, Ic, Id, Ie, If, Ig and Ih in which Lₙ is 2-chloro,4-methoxy, R³ is chlorine and R¹, R² for each compound corresponds to one row of Table A

Table 73

Compounds of the formulae Ia, Ib, Ic, Id, Ie, If, Ig and Ih in which Lₙ is 2-chloro,4-methyl, R³ is chlorine and R¹, R² for each compound corresponds to one row of Table A

Table 74

Compounds of the formulae Ia, Ib, Ic, Id, Ie, If, Ig and Ih in which Lₙ is 2-chloro,4-methoxycarbonyl, R³ is chlorine and R¹, R² for each compound corresponds to one row of Table A

Table 75

Compounds of the formulae Ia, Ib, Ic, Id, Ie, If, Ig and Ih in which Lₙ is 2-chloro,4-bromo, R³ is chlorine and R¹, R² for each compound corresponds to one row of Table A

Table 76

Compounds of the formulae Ia, Ib, Ic, Id, Ie, If, Ig and Ih in which Lₙ is 2-chloro,4-cyano, R³ is chlorine and R¹, R² for each compound corresponds to one row of Table A

Table 77

Compounds of the formulae Ia, Ib, Ic, Id, Ie, If, Ig and Ih in which Lₙ is 2,6-difluoro,4-methoxy, R³ is chlorine and R¹, R² for each compound corresponds to one row of Table A

Table 78

Compounds of the formulae Ia, Ib, Ic, Id, Ie, If, Ig and Ih in which Lₙ is 2-fluoro,3-methyl, R³ is chlorine and R¹, R² for each compound corresponds to one row of Table A

Table 79

Compounds of the formulae Ia, Ib, Ic, Id, Ie, If, Ig and Ih in which Lₙ is 2,5-dimethyl, R³ is chlorine and R¹, R² for each compound corresponds to one row of Table A

Table 80

Compounds of the formulae Ia, Ib, Ic, Id, Ie, If, Ig and Ih in which Lₙ is 2-methyl,4-cyano, R³ is chlorine and R¹, R² for each compound corresponds to one row of Table A

Table 81

Compounds of the formulae Ia, Ib, Ic, Id, Ie, If, Ig and Ih in which Lₙ is 2-methyl,4-bromo, R³is chlorine and R¹, R² for each compound corresponds to one row of Table A

Table 82

Compounds of the formulae Ia, Ib, Ic, Id, Ie, If, Ig and Ih in which Lₙ is 2-methyl,5-fluoro, R³ is chlorine and R¹, R² for each compound corresponds to one row of Table A

Table 83

Compounds of the formulae Ia, Ib, Ic, Id, Ie, If, Ig and Ih in which Lₙ is 2-methyl,4-methoxy, R³is chlorine and R¹, R² for each compound corresponds to one row of Table A

Table 84

Compounds of the formulae Ia, Ib, Ic, Id, Ie, If, Ig and Ih in which Lₙ is 2-methyl,4-methoxycarbonyl, R³ is chlorine and R¹, R² for each compound corresponds to one row of Table A

Table 85

Compounds of the formulae Ia, Ib, Ic, Id, Ie, If, Ig and Ih in which Lₙ is 2,5-dimethyl,4-bromo, R³is chlorine and R¹, R² for each compound corresponds to one row of Table A

Table 86

Compounds of the formulae Ia, Ib, Ic, Id, Ie, If, Ig and Ih in which Lₙ is 2-fluoro,4-bromo, R³ is chlorine and R¹, R² for each compound corresponds to one row of Table A

Table 87

Compounds of the formulae Ia, Ib, Ic, Id, Ie, If, Ig and Ih in which Lₙ is 2-fluoro,4-methoxy, R³is chlorine and R¹, R² for each compound corresponds to one row of Table A

Table 88

Compounds of the formulae Ia, Ib, Ic, Id, Ie, If, Ig and Ih in which Lₙ is 2-fluoro,5-methyl, R³ is chlorine and R¹, R² for each compound corresponds to one row of Table A

Table 89

Compounds of the formulae Ia, Ib, Ic, Id, Ie, If, Ig and Ih in which Lₙ is pentafluoro, R³ is chlorine and R¹, R² for each compound corresponds to one row of Table A

Table 90

Compounds of the formulae Ia, Ib, Ic, Id, Ie, If, Ig and Ih in which Lₙ is 2-fluoro,6-chloro, R³ is bromine and R¹, R² for each compound corresponds to one row of Table A

Table 91

Compounds of the formulae Ia, Ib, Ic, Id, Ie, If, Ig and Ih in which Lₙ is 2,6-difluoro, R³ is bromine and R¹, R² for each compound corresponds to one row of Table A

Table 92

Compounds of the formulae Ia, Ib, Ic, Id, Ie, If, Ig and Ih in which Lₙ is 2,6-dichloro, R³ is bromine and R¹, R² for each compound corresponds to one row of Table A

Table 93

Compounds of the formulae Ia, Ib, Ic, Id, Ie, If, Ig and Ih in which Lₙ is 2-fluoro,6-methyl, R³ is bromine and R¹, R² for each compound corresponds to one row of Table A

Table 94

Compounds of the formulae Ia, Ib, Ic, Id, Ie, If, Ig and Ih in which Lₙ is 2,4,6-trifluoro, R³ is bromine and R¹, R² for each compound corresponds to one row of Table A

Table 95

Compounds of the formulae Ia, Ib, Ic, Id, Ie, If, Ig and Ih in which Lₙ is 2-methyl,4-fluoro, R³is bromine and R¹, R² for each compound corresponds to one row of Table A

Table 96

Compounds of the formulae Ia, Ib, Ic, Id, Ie, If, Ig and Ih in which Lₙ is 2-fluoro,4-methoxycarbonyl, R³ is bromine and R¹, R² for each compound corresponds to one row of Table A

Table 97

Compounds of the formulae Ia, Ib, Ic, Id, Ie, If, Ig and Ih in which Lₙ is 2-fluoro,4-CN, R³ is bromine and R¹, R² for each compound corresponds to one row of Table A

Table 98

Compounds of the formulae Ia, Ib, Ic, Id, Ie, If, Ig and Ih in which Lₙ is 2,4,5-trifluoro, R³ is bromine and R¹, R² for each compound corresponds to one row of Table A

Table 99

Compounds of the formulae Ia, Ib, Ic, Id, Ie, If, Ig and Ih in which Lₙ is 2,4-dichloro, R³ is bromine and R¹, R² for each compound corresponds to one row of Table A

Table 100

Compounds of the formulae Ia, Ib, Ic, Id, Ie, If, Ig and Ih in which Lₙ is 2-chloro, R³ is bromine and R¹, R² for each compound corresponds to one row of Table A

Table 101

Compounds of the formulae Ia, Ib, Ic, Id, Ie, If, Ig and Ih in which Lₙ is 2-fluoro, R³ is bromine and R¹, R² for each compound corresponds to one row of Table A

Table 102

Compounds of the formulae Ia, Ib, Ic, Id, Ie, If, Ig and Ih in which Lₙ is 2,4-difluoro, R³ is bromine and R¹, R² for each compound corresponds to one row of Table A

Table 103

Compounds of the formulae Ia, Ib, Ic, Id, Ie, If, Ig and Ih in which Lₙ is 2-fluoro-4-chloro, R³ is bromine and R¹, R² for each compound corresponds to one row of Table A

Table 104

Compounds of the formulae Ia, Ib, Ic, Id, Ie, If, Ig and Ih in which Lₙ is 2-chloro-4-fluoro, R³ is bromine and R¹, R² for each compound corresponds to one row of Table A

Table 105

Compounds of the formulae Ia, Ib, Ic, Id, Ie, If, Ig and Ih in which Lₙ is 2,3-difluoro, R³ is bromine and R¹, R² for each compound corresponds to one row of Table A

Table 106

Compounds of the formulae Ia, Ib, Ic, Id, Ie, If, Ig and Ih in which Lₙ is 2,5-difluoro, R³ is bromine and R¹, R² for each compound corresponds to one row of Table A

Table 107

Compounds of the formulae Ia, Ib, Ic, Id, Ie, If, Ig and Ih in which Lₙ is 2,3,4-trifluoro, R³ is bromine and R¹, R² for each compound corresponds to one row of Table A

Table 108

Compounds of the formulae Ia, Ib, Ic, Id, Ie, If, Ig and Ih in which Lₙ is 2-methyl, R³ is bromine and R¹, R² for each compound corresponds to one row of Table A

Table 109

Compounds of the formulae Ia, Ib, Ic, Id, Ie, If, Ig and Ih in which Lₙ is 2,4-dimethyl, R³ is bromine and R¹, R² for each compound corresponds to one row of Table A

Table 110

Compounds of the formulae Ia, Ib, Ic, Id, Ie, If, Ig and Ih in which Lₙ is 2-methyl-4-chloro, R³is bromine and R¹, R² for each compound corresponds to one row of Table A

Table 111

Compounds of the formulae Ia, Ib, Ic, Id, Ie, If, Ig and Ih in which Lₙ is 2-fluoro-4-methyl, R³is bromine and R¹, R² for each compound corresponds to one row of Table A

Table 112

Compounds of the formulae Ia, Ib, Ic, Id, Ie, If, Ig and Ih in which Lₙ is 2,6-dimethyl, R³ is bromine and R¹, R² for each compound corresponds to one row of Table A

Table 113

Compounds of the formulae Ia, Ib, Ic, Id, Ie, If, Ig and Ih, in which Lₙ is 2,4,6-trimethyl, R³ is bromine and R¹, R² for each compound corresponds to one row of Table A

Table 114

Compounds of the formulae Ia, Ib, Ic, Id, Ie, If, Ig and Ih in which Lₙ is 2,6-difluoro-4-cyano, R³is bromine and R¹, R² for each compound corresponds to one row of Table A

Table 115

Compounds of the formulae Ia, Ib, Ic, Id, Ie, If, Ig and Ih in which Lₙ is 2,6-difluoro-4-methyl, R³is bromine and R¹, R² for each compound corresponds to one row of Table A

Table 116

Compounds of the formulae Ia, Ib, Ic, Id, Ie, If, Ig and Ih in which Lₙ is 2,6-difluoro4-methoxycarbonyl, R³ is bromine and R¹, R² for each compound corresponds to one row of Table A

Table 117

Compounds of the formulae Ia, Ib, Ic, Id, Ie, If, Ig and Ih in which Lₙ is 2-chloro,4-methoxy, R³is bromine and R¹, R² for each compound corresponds to one row of Table A

Table 118

Compounds of the formulae Ia, Ib, Ic, Id, Ie, If, Ig and Ih in which Lₙ is 2-chloro,4-methyl, R³is bromine and R¹, R² for each compound corresponds to one row of Table A

Table 119

Compounds of the formulae Ia, Ib, Ic, Id, Ie, If, Ig and Ih in which Lₙ is 2-chloro,4-methoxycarbonyl, R³is bromine and R¹, R² for each compound corresponds to one row of Table A

Table 120

Compounds of the formulae Ia, Ib, Ic, Id, Ie, If, Ig and Ih in which Lₙ is 2-chloro,4-bromo, R³is bromine and R¹, R² for each compound corresponds to one row of Table A

Table 121

Compounds of the formulae Ia, Ib, Ic, Id, Ie, If, Ig and Ih in which Lₙ is 2-chloro,4-cyano, R³ is bromine and R¹, R² for each compound corresponds to one row of Table A

Table 122

Compounds of the formulae Ia, Ib, Ic, Id, Ie, If, Ig and Ih in which Lₙ is 2,6-difluoro,4-methoxy, R³is bromine and R¹, R² for each compound corresponds to one row of Table A

Table 123

Compounds of the formulae Ia, Ib, Ic, Id, Ie, If, Ig and Ih in which Lₙ is 2-fluoro,3-methyl, R³is bromine and R¹, R² for each compound corresponds to one row of Table A

Table 124

Compounds of the formulae Ia, Ib, Ic, Id, Ie, If, Ig and Ih in which Lₙ is 2,5-dimethyl, R³ is bromine and R¹, R² for each compound corresponds to one row of Table A

Table 125

Compounds of the formulae Ia, Ib, Ic, Id, Ie, If, Ig and Ih in which Lₙ is 2-methyl,4-cyano, R³ is bromine and R¹, R² for each compound corresponds to one row of Table A

Table 126

Compounds of the formulae Ia, Ib, Ic, Id, Ie, If, Ig and Ih in which Lₙ is 2-methyl,4-bromo, R³ is bromine and R¹, R² for each compound corresponds to one row of Table A

Table 127

Compounds of the formulae Ia, Ib, Ic, Id, Ie, If, Ig and Ih in which Lₙ is 2-methyl,5-fluoro, R³ is bromine and R¹, R² for each compound corresponds to one row of Table A

Table 128

Compounds of the formulae Ia, Ib, Ic, Id, Ie, If, Ig and Ih in which Lₙ is 2-methyl,4-methoxy, R³ is bromine and R¹, R² for each compound corresponds to one row of Table A

Table 129

Compounds of the formulae Ia, Ib, Ic, Id, Ie, If, Ig and Ih, in which Lₙ is 2-methyl,4-methoxycarbonyl, R³ is bromine and R¹, R² for each compound corresponds to one row of Table A

Table 130

Compounds of the formulae Ia, Ib, Ic, Id, Ie, If, Ig and Ih in which Lₙ is 2,5-dimethyl,4-bromo, R³ is bromine and R¹, R² for each compound corresponds to one row of Table A

Table 131

Compounds of the formulae Ia, Ib, Ic, Id, Ie, If, Ig and Ih in which Lₙ is 2-fluoro,4-bromo, R³ is bromine and R¹, R² for each compound corresponds to one row of Table A

Table 132

Compounds of the formulae Ia, Ib, Ic, Id, Ie, If, Ig and Ih in which Lₙ is 2-fluoro,4-methoxy, R³ is bromine and R¹, R² for each compound corresponds to one row of Table A

Table 133

Compounds of the formulae Ia, Ib, Ic, Id, Ie, If, Ig and Ih in which Lₙ is 2-fluoro,5-methyl, R³is bromine and R¹, R² for each compound corresponds to one row of Table A

Table 134

Compounds of the formulae Ia, Ib, Ic, Id, Ie, If, Ig and Ih in which Lₙ is pentafluoro, R³ is bromine and R¹, R² for each compound corresponds to one row of Table A

Table 135

Compounds of the formulae Ia, Ib, Ic, Id, Ie, If, Ig and Ih in which Lₙ is 2-fluoro,6-chloro, R³is cyano and R¹, R² for each compound corresponds to one row of Table A

Table 136

Compounds of the formulae Ia, Ib, Ic, Id, Ie, If, Ig and Ih in which Lₙ is 2,6-difluoro, R³ is cyano and R¹, R² for each compound corresponds to one row of Table A

Table 137

Compounds of the formulae Ia, Ib, Ic, Id, Ie, If, Ig and Ih in which Lₙ is 2,6-dichloro, R³ is cyano and R¹, R² for each compound corresponds to one row of Table A

Table 138

Compounds of the formulae Ia, Ib, Ic, Id, Ie, If, Ig and Ih in which Lₙ is 2-fluoro,6-methyl, R³ is cyano and R¹, R² for each compound corresponds to one row of Table A

Table 139

Compounds of the formulae Ia, Ib, Ic, Id, Ie, If, Ig and Ih in which Lₙ is 2,4,6-trifluoro, R³ is cyano and R¹, R² for each compound corresponds to one row of Table A

Table 140

Compounds of the formulae Ia, Ib, Ic, Id, Ie, If, Ig and Ih in which Lₙ is 2-methyl,4-fluoro, R³is cyano and R¹, R² for each compound corresponds to one row of Table A

Table 141

Compounds of the formulae Ia, Ib, Ic, Id, Ie, If, Ig and Ih in which Lₙ is 2-fluoro,4-methoxycarbonyl, R³ is cyano and R¹, R² for each compound corresponds to one row of Table A

Table 142

Compounds of the formulae Ia, Ib, Ic, Id, Ie, If, Ig and Ih in which Lₙ is 2-fluoro,4-CN, R³ is cyano and R¹, R² for each compound corresponds to one row of Table A

Table 143

Compounds of the formulae Ia, Ib, Ic, Id, Ie, If, Ig and Ih in which Lₙ is 2,4,5-trifluoro, R³ is cyano and R¹, R² for each compound corresponds to one row of Table A

Table 144

Compounds of the formulae Ia, Ib, Ic, Id, Ie, If, Ig and Ih in which Lₙ is 2,4-dichloro, R³ is cyano and R¹, R² for each compound corresponds to one row of Table A

Table 145

Compounds of the formulae Ia, Ib, Ic, Id, Ie, If, Ig and Ih, in which Lₙ is 2-chloro, R³ is cyano and R¹, R² for each compound corresponds to one row of Table A

Table 146

Compounds of the formulae Ia, Ib, Ic, Id, Ie, If, Ig and Ih in which Lₙ is 2-fluoro, R³ is cyano and R¹, R² for each compound corresponds to one row of Table A

Table 147

Compounds of the formulae Ia, Ib, Ic, Id, Ie, If, Ig and Ih in which Lₙ is 2,4-difluoro, R³ is cyano and R¹, R² for each compound corresponds to one row of Table A

Table 148

Compounds of the formulae Ia, Ib, Ic, Id, Ie, If, Ig and Ih in which Lₙ is 2-fluoro4-chloro, R³ is cyano and R¹, R² for each compound corresponds to one row of Table A

Table 149

Compounds of the formulae Ia, Ib, Ic, Id, Ie, If, Ig and Ih in which Lₙ is 2-chloro-4-fluoro, R³ is cyano and R¹, R² for each compound corresponds to one row of Table A

Table 150

Compounds of the formulae Ia, Ib, Ic, Id, Ie, If, Ig and Ih in which Lₙ is 2,3-difluoro, R³ is cyano and R¹, R² for each compound corresponds to one row of Table A

Table 151

Compounds of the formulae Ia, Ib, Ic, Id, Ie, If, Ig and Ih in which Lₙ is 2,5-difluoro, R³ is cyano and R¹, R² for each compound corresponds to one row of Table A

Table 152

Compounds of the formulae Ia, Ib, Ic, Id, Ie, If, Ig and Ih in which Lₙ is 2,3,4-trifluoro, R³ is cyano and R¹, R² for each compound corresponds to one row of Table A

Table 153

Compounds of the formulae Ia, Ib, Ic, Id, Ie, If, Ig and Ih in which Lₙ is 2-methyl, R³ is cyano and R¹, R² for each compound corresponds to one row of Table A

Table 154

Compounds of the formulae Ia, Ib, Ic, Id, Ie, If, Ig and Ih in which Lₙ is 2,4-dimethyl, R³ is cyano and R¹, R² for each compound corresponds to one row of Table A

Table 155

Compounds of the formulae Ia, Ib, Ic, Id, Ie, If, Ig and Ih in which Lₙ is 2-methyl-4-chloro, R³ is cyano and R¹, R² for each compound corresponds to one row of Table A

Table 156

Compounds of the formulae Ia, Ib, Ic, Id, Ie, If, Ig and Ih in which Lₙ is 2-fluoro-4-methyl, R³ is cyano and R¹, R² for each compound corresponds to one row of Table A

Table 157

Compounds of the formulae Ia, Ib, Ic, Id, Ie, If, Ig and Ih in which Lₙ is 2,6-dimethyl, R³ is cyano and R¹, R² for each compound corresponds to one row of Table A

Table 158

Compounds of the formulae Ia, Ib, Ic, Id, Ie, If, Ig and Ih in which Lₙ is 2,4,6-trimethyl, R³ is cyano and R¹, R² for each compound corresponds to one row of Table A

Table 159

Compounds of the formulae Ia, Ib, Ic, Id, Ie, If, Ig and Ih in which Lₙ is 2,6-difluoro-4-cyano, R³ is cyano and R¹, R² for each compound corresponds to one row of Table A

Table 160

Compounds of the formulae Ia, Ib, Ic, Id, Ie, If, Ig and Ih in which Lₙ is 2,6-difluoro-4-methyl, R³ is cyano and R¹, R² for each compound corresponds to one row of Table A

Table 161

Compounds of the formulae Ia, Ib, Ic, Id, Ie, If, Ig and Ih in which Lₙ is 2,6-difluoro-4-methoxycarbonyl, R³ is cyano and R¹, R² for each compound corresponds to one row of Table A

Table 162

Compounds of the formulae Ia, Ib, Ic, Id, Ie, If, Ig and Ih in which Lₙ is 2-chloro,4-methoxy, R³ is cyano and R¹, R² for each compound corresponds to one row of Table A

Table 163

Compounds of the formulae Ia, Ib, Ic, Id, Ie, If, Ig and Ih in which Lₙ is 2-chloro,4-methyl, R³ is cyano and R¹, R² for each compound corresponds to one row of Table A

Table 164

Compounds of the formulae Ia, Ib, Ic, Id, Ie, If, Ig and Ih in which Lₙ is 2-chloro,4-methoxycarbonyl, R³ is cyano and R¹, R² for each compound corresponds to one row of Table A

Table 165

Compounds of the formulae Ia, Ib, Ic, Id, Ie, If, Ig and Ih in which Lₙ is 2-chloro,4-bromo, R³ is cyano and R¹, R² for each compound corresponds to one row of Table A

Table 166

Compounds of the formulae Ia, Ib, Ic, Id, Ie, If, Ig and Ih in which Lₙ is 2-chloro,4-cyano, R³ is cyano and R¹, R² for each compound corresponds to one row of Table A

Table 167

Compounds of the formulae Ia, Ib, Ic, Id, Ie, If, Ig and Ih in which Lₙ is 2,6-difluoro,4-methoxy, R³ is cyano and R¹, R² for each compound corresponds to one row of Table A

Table 168

Compounds of the formulae Ia, Ib, Ic, Id, Ie, If, Ig and Ih in which Lₙ is 2-fluoro,3-methyl, R³ is cyano and R¹, R² for each compound corresponds to one row of Table A

Table 169

Compounds of the formulae Ia, Ib, Ic, Id, Ie, If, Ig and Ih in which Lₙ is 2,5-dimethyl, R³ is cyano and R¹, R² for each compound corresponds to one row of Table A

Table 170

Compounds of the formulae Ia, Ib, Ic, Id, Ie, If, Ig and Ih in which Lₙ is 2-methyl,4-cyano, R³ is cyano and R¹, R² for each compound corresponds to one row of Table A

Table 171

Compounds of the formulae Ia, Ib, Ic, Id, Ie, If, Ig and Ih in which Lₙ is 2-methyl,4-bromo, R³ is cyano and R¹, R² for each compound corresponds to one row of Table A

Table 172

Compounds of the formulae Ia, Ib, Ic, Id, Ie, If, Ig and Ih in which Lₙ is 2-methyl,5-fluoro, R³ is cyano and R¹, R² for each compound corresponds to one row of Table A

Table 173

Compounds of the formulae Ia, Ib, Ic, Id, Ie, If, Ig and Ih in which Lₙ is 2-methyl,4-methoxy, R³ is cyano and R¹, R² for each compound corresponds to one row of Table A

Table 174

Compounds of the formulae Ia, Ib, Ic, Id, Ie, If, Ig and Ih in which Lₙ is 2-methyl,4-methoxycarbonyl, R³ is cyano and R¹, R² for each compound corresponds to one row of Table A

Table 175

Compounds of the formulae Ia, Ib, Ic, Id, Ie, If, Ig and Ih in which Lₙ is 2,5-dimethyl,4-bromo, R³ is cyano and R¹, R² for each compound corresponds to one row of Table A

Table 176

Compounds of the formulae Ia, Ib, Ic, Id, Ie, If, Ig and Ih in which Lₙ is 2-fluoro,4-bromo, R³is cyano and R¹, R² for each compound corresponds to one row of Table A

Table 177

Compounds of the formulae Ia, Ib, Ic, Id, Ie, If, Ig and Ih in which Lₙ is 2-fluoro,4-methoxy, R³ is cyano and R¹, R² for each compound corresponds to one row of Table A

Table 178

Compounds of the formulae Ia, Ib, Ic, Id, Ie, If, Ig and Ih in which Lₙ is 2-fluoro,5-methyl, R³ is cyano and R¹, R² for each compound corresponds to one row of Table A

Table 179

Compounds of the formulae Ia, Ib, Ic, Id, Ie, If, Ig and Ih in which Lₙ is pentafluoro, R³ is cyano and R¹, R² for each compound corresponds to one row of Table A

TABLE A

| No. | R¹ | R² |
|---|---|---|
| A-1 | CH₂CH₃ | H |
| A-2 | CH₂CH₃ | CH₃ |
| A-3 | CH₂CH₃ | CH₂CH₃ |
| A-4 | CH₂CH₂CH₃ | H |
| A-5 | CH₂CH₂CH₃ | CH₃ |
| A-6 | CH₂CH₂CH₃ | CH₂CH₃ |
| A-7 | CH₂CH₂CH₃ | CH₂CH₂CH₃ |
| A-8 | CH₂CH₂F | H |
| A-9 | CH₂CH₂F | CH₃ |
| A-10 | CH₂CH₂F | CH₂CH₃ |
| A-11 | CH₂CF₃ | H |
| A-12 | CH₂CF₃ | CH₃ |
| A-13 | CH₂CF₃ | CH₂CH₃ |
| A-14 | CH₂CF₃ | CH₂CH₂CH₃ |
| A-15 | CH₂CCl₃ | H |
| A-16 | CH₂CCl₃ | CH₃ |
| A-17 | CH₂CCl₃ | CH₂CH₃ |
| A-18 | CH₂CCl₃ | CH₂CH₂CH₃ |
| A-19 | CH(CH₃)₂ | H |
| A-20 | CH(CH₃)₂ | CH₃ |
| A-21 | CH(CH₃)₂ | CH₂CH₃ |
| A-22 | CH(CH₃)₂ | CH₂CH₂CH₃ |
| A-23 | CH₂C(CH₃)₃ | H |
| A-24 | CH₂C(CH₃)₃ | CH₃ |
| A-25 | CH₂C(CH₃)₃ | CH₂CH₃ |
| A-26 | CH₂CH(CH₃)₂ | H |
| A-27 | CH₂CH(CH₃)₂ | CH₃ |
| A-28 | CH₂CH(CH₃)₂ | CH₂CH₃ |
| A-29 | (±) CH(CH₂CH₃)CH₃ | H |
| A-30 | (±) CH(CH₂CH₃)CH₃ | CH₃ |
| A-31 | (±) CH(CH₂CH₃)CH₃ | CH₂CH₃ |
| A-32 | (R) CH(CH₂CH₃)CH₃ | H |
| A-33 | (R) CH(CH₂CH₃)CH₃ | CH₃ |
| A-34 | (R) CH(CH₂CH₃)CH₃ | CH₂CH₃ |
| A-35 | (S) CH(CH₂CH₃)CH₃ | H |
| A-36 | (S) CH(CH₂CH₃)CH₃ | CH₃ |
| A-37 | (S) CH(CH₂CH₃)CH₃ | CH₂CH₃ |
| A-38 | (±) CH(CH₃)—CH(CH₃)₂ | H |
| A-39 | (±) CH(CH₃)—CH(CH₃)₂ | CH₃ |
| A-40 | (±) CH(CH₃)—CH(CH₃)₂ | CH₂CH₃ |
| A-41 | (R) CH(CH₃)—CH(CH₃)₂ | H |
| A-42 | (R) CH(CH₃)—CH(CH₃)₂ | CH₃ |
| A-43 | (R) CH(CH₃)—CH(CH₃)₂ | CH₂CH₃ |
| A-44 | (S) CH(CH₃)—CH(CH₃)₂ | H |
| A-45 | (S) CH(CH₃)—CH(CH₃)₂ | CH₃ |
| A-46 | (S) CH(CH₃)—CH(CH₃)₂ | CH₂CH₃ |
| A-47 | (±) CH(CH₃)—C(CH₃)₃ | H |
| A-48 | (±) CH(CH₃)—C(CH₃)₃ | CH₃ |
| A-49 | (±) CH(CH₃)—C(CH₃)₃ | CH₂CH₃ |
| A-50 | (R) CH(CH₃)—C(CH₃)₃ | H |
| A-51 | (R) CH(CH₃)—C(CH₃)₃ | CH₃ |
| A-52 | (R) CH(CH₃)—C(CH₃)₃ | CH₂CH₃ |
| A-53 | (S) CH(CH₃)—C(CH₃)₃ | H |
| A-54 | (S) CH(CH₃)—C(CH₃)₃ | CH₃ |
| A-55 | (S) CH(CH₃)—C(CH₃)₃ | CH₂CH₃ |
| A-56 | (±) CH(CH₃)—CF₃ | H |
| A-57 | (±) CH(CH₃)—CF₃ | CH₃ |
| A-58 | (±) CH(CH₃)—CF₃ | CH₂CH₃ |
| A-59 | (R) CH(CH₃)—CF₃ | H |
| A-60 | (R) CH(CH₃)—CF₃ | CH₃ |
| A-61 | (R) CH(CH₃)—CF₃ | CH₂CH₃ |
| A-62 | (S) CH(CH₃)—CF₃ | H |
| A-63 | (S) CH(CH₃)—CF₃ | CH₃ |
| A-64 | (S) CH(CH₃)—CF₃ | CH₂CH₃ |
| A-65 | (±) CH(CH₃)—CCl₃ | H |
| A-66 | (±) CH(CH₃)—CCl₃ | CH₃ |
| A-67 | (±) CH(CH₃)—CCl₃ | CH₂CH₃ |
| A-68 | (R) CH(CH₃)—CCl₃ | H |
| A-69 | (R) CH(CH₃)—CCl₃ | CH₃ |
| A-70 | (R) CH(CH₃)—CCl₃ | CH₂CH₃ |
| A-71 | (S) CH(CH₃)—CCl₃ | H |
| A-72 | (S) CH(CH₃)—CCl₃ | CH₃ |
| A-73 | (S) CH(CH₃)—CCl₃ | CH₂CH₃ |
| A-74 | CH₂C(CH₃)═CH₂ | H |
| A-75 | CH₂C(CH₃)═CH₂ | CH₃ |
| A-76 | CH₂C(CH₃)═CH₂ | CH₂CH₃ |
| A-77 | Cyclopentyl | H |
| A-78 | Cyclopentyl | CH₃ |
| A-79 | Cyclopentyl | CH₂CH₃ |

| | |
|---|---|
| A-80 | —(CH₂)₄— |
| A-81 | (±) —(CH₂)₂—CH(CH₃)—CH₂— |
| A-82 | (R) —(CH₂)₂—CH(CH₃)—CH₂— |
| A-83 | (S) —(CH₂)₂—CH(CH₃)—CH₂— |
| A-84 | —(CH₂)₂—CH(OCH₃)—CH₂— |
| A-85 | —(CH₂)₂—CH(CH₂CH₃)—CH₂— |
| A-86 | —(CH₂)₂—CH[CH(CH₃)₂]—CH₂— |
| A-87 | (±) —(CH₂)₃—CH(CH₃)— |
| A-88 | (±) —CH(CH₃)—(CH₂)₂—CH(CH₃)— |
| A-89 | —CH₂—CH═CH—CH₂— |
| A-90 | —(CH₂)₅— |
| A-91 | (±) —(CH₂)₄—CH(CH₃)— |
| A-92 | —(CH₂)₂—CH(CH₃)—(CH₂)₂— |
| A-93 | (±) —(CH₂)₃—CH(CH₃)—CH₂— |
| A-94 | (R) —(CH₂)₃—CH(CH₃)—CH₂— |
| A-95 | (S) —(CH₂)₃—CH(CH₃)—CH₂— |
| A-96 | —(CH₂)₂—C(O[CH₂]₂O)—(CH₂)₂— |
| A-97 | —(CH₂)₂—C(O[CH₂]₃O)—(CH₂)₂— |
| A-98 | 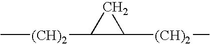 |
| A-99 | —(CH₂)₂—CH═CH—CH₂— |

The compounds I are suitable as fungicides. They are distinguished through an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines.
Erysiphe cichoracearum and Sphaerotheca fuliginea on cucurbits,
Fusarium and Verticillium species on various plants,
Mycosphaerella species on bananas and peanuts,
Phytophthora infestants on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (for example wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the desired effect. Amounts customarily applied in the protection of materials, are for example, 0.001 g to 2 kg, preferably 0.005 g to 1 kg, of active compound per cubic meter of treated material.

The compounds I can be converted to the customary formulations, for example solutions, emulsions, suspensions, dusts, powders, pastes and granules. The application form depends on the particular intended use; it should in any case ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations:

1. Products for dilution with water

A) Water-soluble concentrates (SL)

10 parts by weight of a compound according to the invention are dissolved in water or in a water-soluble solvent. As an alternative, wetters or other auxiliaries are added. The active compound dissolves upon dilution with water.

B) Dispersible concentrates (DC)

20 parts by weight of a compound according to the invention are dissolved in cyclohexanone with addition of a dispersant, for example polyvinylpyrrolidone. Dilution with water gives a dispersion.

C) Emulsifiable concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

D) Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturax) and made into a homogeneous emulsion. Dilution with water gives an emulsion.

E) Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

F) Water-dispersible granules and water-soluble granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G) Water-dispersible powders and water-soluble powders (WP, SP)

75 parts by weight of a compound according to the invention are ground in a rotor-stator mill with addition of dispersants, wetters and silica gel. Dilution with water gives a stable dispersion or solution of the active compound.
2. Products to be applied undiluted

H) Dustable powders (DP)

5 parts by weight of a compound according to the invention are ground finely and mixed intimately with 95% of finely divided kaolin. This gives a dustable product.

I) Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

J) ULV solutions (UL)

10 parts by weight of a compound according to the invention are dissolved in an organic solvent, for example xylene. This gives a product to be applied undiluted.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should always ensure the finest possible dispersion of the active compounds according to the invention.

Aqueous application forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if need be also not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following lists of fungicides, with which the compounds according to the invention can be used in conjunction, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidine, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil, sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

### Synthesis of the Hydrazide Intermediates

### Example 1

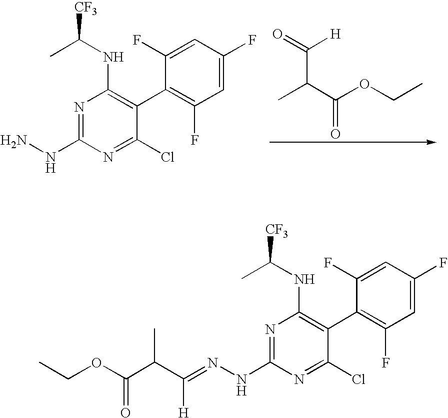

1.9 g (5 mmol) of the hydrazide were initially charged in 40 ml of diethyl ether. 0.8 g (5.5 mmol) of the aldehyde were then added, and the mixture was stirred at room temperature overnight. The reaction was monitored by TLC. After removal of the solvent in a rotary evaporator, the product was purified by column chromatography (DCM). The product was obtained as beige crystals. Yield: 64%. ¹H-NMR (CDCl₃)=1.2-1.3 (bm, 6 H); 1.4 (d, 3 H); 3.4 (m, 1 H); 4.2 (q, 2 H); 4.25 (d, 1 H, NH); 6.8 (m, 2 H); 7.3 (d, 1 H); 8.2 (bs, 1 H).

### Example 2

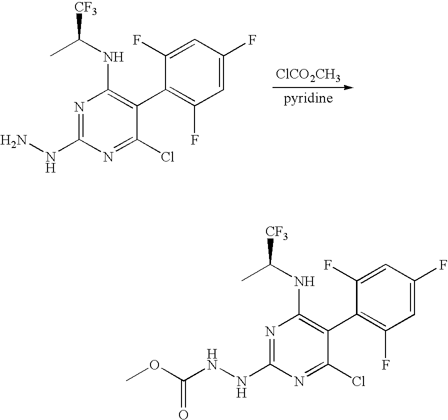

1.0 g (2.6 mmol) of the hydrazide was dissolved in 1 ml of absolute pyridine. 0.25 g (2.6 mmol) of methyl chloroformate and 5 ml of water were then added. The mixture was stirred overnight. The reaction was monitored by HPLC. For work-up, the solid was filtered off with suction and washed successively with 1.5 ml of dist. water, twice with 10% strength acetic acid and finely three times with water. Drying gave 0.9 g of the product (75% yield).

### Examples of Active Compounds

### Example 3

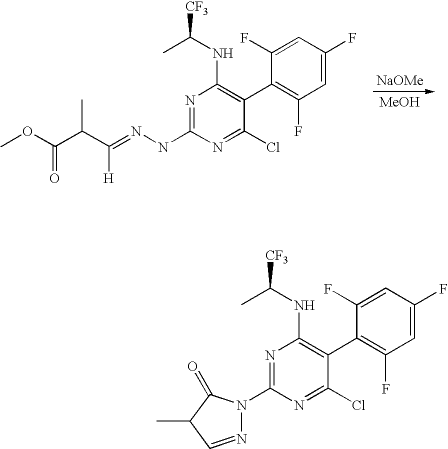

1.4 g (3 mmol) of the hydrazone (Example 1) were dissolved in 10 ml of absolute methanol. 0.6 g (3 mmol) of sodium methoxide solution (30% in methanol) was added, and the mixture was then stirred at room temperature overnight. The reaction was monitored by HPLC. After removal of the solvent in a rotary evaporator, the reaction mixture was stirred with distilled water and adjusted to pH 1-2 using 5% strength hydrochloric acid. The mixture was extracted three times with DCM and the extracts were washed once with saturated sodium chloride solution. The combined extracts were dried and concentrated. The residue was digested with diisopropyl ether, filtered off with suction, washed with diisopropyl ether and n-pentane and dried. The product was obtained as a colorless solid. Yield: 0.7 g (55%). ¹H-NMR (CDCl₃) ppm=1.1 (d, 3 H); 1.4 (d, 3 H); 2.0 (s, 3 H); 4.9 (d, 1 H); 5.2 (m, 1 H); 6.9 (m, 2 H); 7.4 (d, 1 H).

### Example 4

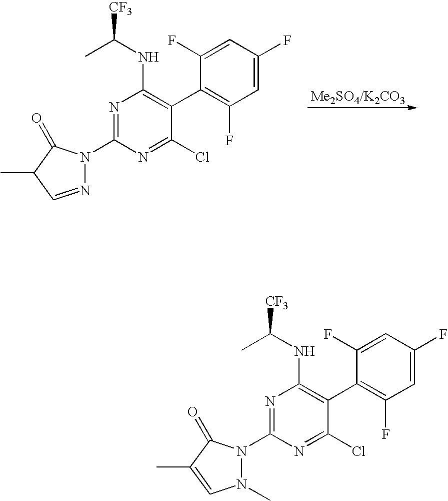

0.25 g of the pyrimidine (Example 3) was dissolved in 5 ml of methanol, and 0.15 g (1.2 mmol) of dimethyl sulfate and 0.14 g (1 mmol) of potassium carbonate was added. The mixture was stirred at RT for three hours. The reaction was monitored by HPLC. More dimethyl sulfate (0.15 g, 1.2 mmol) was added for complete conversion. To destroy excess dimethyl sulfate, the mixture was stirred with 10% strength aqueous ammonia solution and dichloromethane (DCM). After phase separation, the aqueous phase was extracted with DCM. The combined organic phases were washed with water and then dried. The solvent was removed in a rotary evaporator and the product was then purified by column chromatography (toluene: ethyl acetate 9:1; 7:3). Yield: 100 mg (38%). ¹H-NMR (CDCl₃) ppm=1.25 (d, 3 H); 1.8 (s, 3 H); 3.2 (s, 3 H); 5.0 (m, 1 H); 5.1 (m, 1 H); 6.8 (m, 2 H); 7.0 (m, 1 H).

With appropriate modification of the starting materials, the procedures given in the synthesis examples below were used to obtain further compounds 1. The compounds obtained in this manner are listed in Table A below, together with physical data.

TABLE A

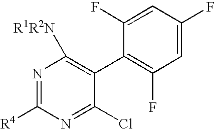

| | | | Phys. |
|---|---|---|---|
| | | | Constants |
| No. | R⁴ | NR¹R² | m.p. [° C.] |
| I-.01 | 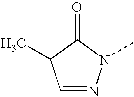 | 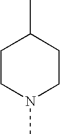 | Oil |
| I-.02 | 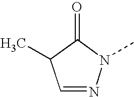 | 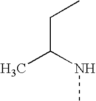 | m.p.: 187-189° C. |
| I-.03 | 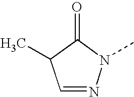 | 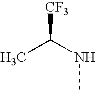 | 217-218 |
| I-.04 | 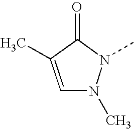 | 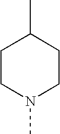 | Oil |
| I-.05 | 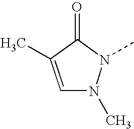 | 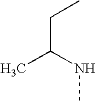 | 215-217 |
| I-.06 | 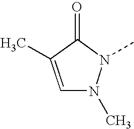 | 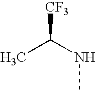 | 194-196 |
| I-.07 | 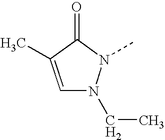 | 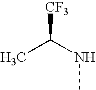 | 207-211 |
| I-.08 | 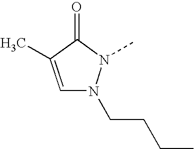 | 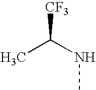 | 200-204 |
| I-.09 | 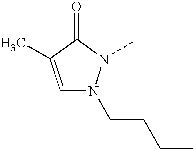 | 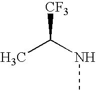 | Oil |
| I-.10 | 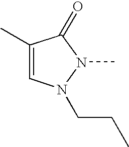 | 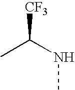 | Oil |
| I-.11 | 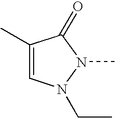 | 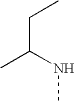 | 155-157 |
| I-.12 | 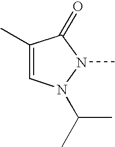 | 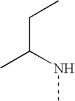 | Oil |
| I-.13 | 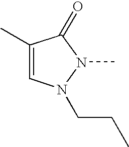 | 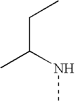 | 179-183 |

### Examples of the Action Against Harmful Fungi

The fungicidal action of the compounds of the formula I was demonstrated by the following experiments:

The active compounds were prepared separately or together as a stock solution with 0.25% by weight of active compound in acetone or DMSO. 1% by weight of the emulsifier Uniperol® EL (wetting agent having emulsifying and dispersing action based on ethoxylated alkylphenols) was added to this solution, and the solution was diluted with water to the desired concentration.

### Use Examples

1. Activity against early blight of tomato caused by Alternaria solani, protective application Leaves of potted plants of the cultivar “Groβpe Fleischtomate St. Pierre” were sprayed to runoff point with an aqueous suspension of the active compound concentration given below. The next day, the leaves were infected with an aqueous spore suspension of Alternaria solani in 2% biomalt solution with a concentration of 0.17×10⁶ spores/ml. The plants were then placed in a water-vapor-saturated chamber at temperatures between 20 and 22° C. After 5 days, the early blight on the untreated but infected control plants had developed to such an extent that the infection could be determined visually in %.

The plants which have been treated with the active compounds according to the invention showed considerably less infection than the untreated plants.

1. A 2-substituted pyrimidine of the formula I 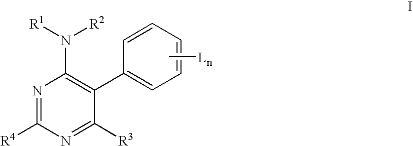
in which the index and the substituents are as defined below:
n is an integer from 1 to 5, where at least one substituent L is located in the ortho-position on the phenyl ring;
L is halogen, cyano, cyanato (OCN), nitro, C₁-C₈-alkyl, C₂-C₁₀-alkenyl, C₂-C₁₀-alkynyl, C₁-C₆-alkoxy, —C(═O)—A, —C(═O)—O—A, —C(═O)—N(A′)A, C(A′)(═N—OA), N(A′)A, N(A′)—C(═O)—A, N(A″)—C(═O)—N(A′)A, S(═O)ₘ—A, S(═O)ₘ—O—A or S(═O)ₘ—N(A′)A,
m is 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated or may be substituted by cyano or C₁-C₄-alkoxy; or A and A′ together with the atoms to which they are attached are a five- or six-membered saturated, partially unsaturated or aromatic heterocycle which contains one to four heteroatoms from the group consisting of O, N and S;
where the aliphatic, alicyclic or aromatic groups of the radical definitions of L for their part may be partially or fully halogenated or may carry one to four groups R^{u}:
R^{u} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)—A, —C(═O)—O—A, —C(═O)—N(A′)A, C(A′)(═N—OA), N(A′)A, N(A′)—C(═O)—A, N(A″)—C(═O)—N(A′)A, S(═O)ₘ—A, S(═O)ₘ—O—A or S(═O)ₘ—N(A′)A, where m, A, A′, A″ are as defined above and where the aliphatic, alicyclic or aromatic groups for their part may be partially or fully halogenated or may carry
one to three groups R^{v}, R^{v} having the same meaning as R^{u};
R¹, R² independently of one another are C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl or C₂-C₆-haloalkynyl;
R² may additionally be hydrogen;
R¹and R² may also, together with the nitrogen atom to which they are attached, form a saturated or unsaturated five- or six-membered ring which may be interrupted by an ether (—O—), carbonyl C[═O]-, thio (—S—), sulfoxyl (—S[═O]—) or sulfenyl (—SO₂—) group;
R³ is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy, where the alkyl, alkenyl and alkynyl radicals of R³ may be substituted by halogen, cyano, nitro, C₁-C₂-alkoxy or C₁-C₄-alkoxycarbonyl;
R⁴ corresponds to one of the formulae 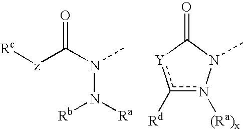
where
x is 0 or 1;
R^{a}, R^{b} and R^{c} independently of one another are hydrogen, C₁-C₆-alkyl, C₂-C₈-alkenyl, C₂-C₈-alkynyl, C₃-C₆-cycloalkyl, C₄-C₆-Cycloalkenyl,
R^{a}, R^{b} together with the nitrogen atom to which they are attached may have the meaning R^{c}—Z—C(R^{d})═N;
Z is oxygen or N—R^{c};
Y is C(H)—R^{e}, C—R^{e}, N—N(H)—R^{c} or N—R^{c′}; [symbol]
may be a double bond or a single bond;
R^{d}, R^{e} have the same meanings as R^{c} and may additionally be halogen or cyano;
R^{d} together with the carbon to which it is attached may be a carbonyl group;
where the aliphatic, alicyclic or aromatic groups of the radical definitions of R^{a}, R^{b}, R^{c}, R^{d} or R^{e} for their part may be partially or fully halogenated or may carry one to four groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, and where two of the radicals R^{a}, R^{b} or R^{c} together with the atoms to which they are attached may form a five- or six-membered saturated, partially unsaturated or aromatic heterocycle which contains one to four heteroatoms from the group consisting of O, N and S.

2. The 2-substituted pyrimidine according to claim 1 where R³ is chlorine, cyano, methyl, ethyl or bromine.

3. The 2-substituted pyrimidine according to claim 1 where R⁴ is one of the formulae

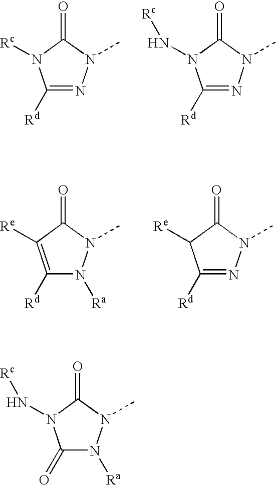

4. The 2-substituted pyrimidine according to claim 1 where R⁴ corresponds to the formula

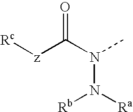

5. The 2-substituted pyrimidine according to claim 1 which the phenyl group substituted by Lₙ is the group B 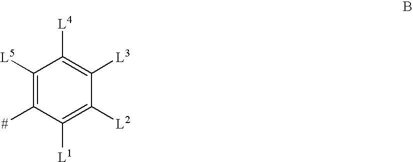
where # is the point of attachment to the pyrimidine skeleton and
L¹ is fluorine, chlorine, CH₃ or CF₃;
L², L⁴ independently of one another are hydrogen, CH₃ or fluorine;
L³ is hydrogen, fluorine, chlorine, cyano, CH₃, SCH₃, OCH₃, SO₂CH₃, NH—C(═O)CH₃, N(CH₃)—C(═O)CH₃ or COOCH₃ and
L⁵ is hydrogen, fluorine, chlorine or CH₃.

6. A process for preparing 2-substituted pyrimidines of the formula I according to claim 3 where R⁴ is a pyrazolone, which comprises condensing a compound of the formula II 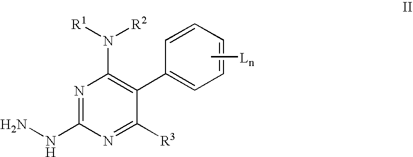
in which the substituents L, R¹, R² and R³ are with a 1,3-dicarbonyl compound of the formula III 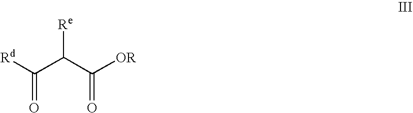
in which R is a C₁-C₆-alkyl radical, and then cyclizing the resulting compound IV 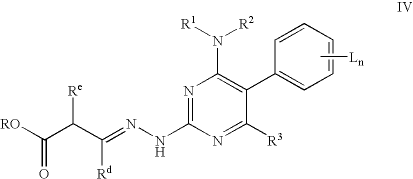
with a base to give IA 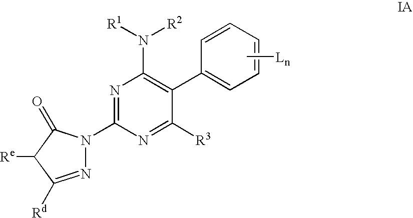
which is, if appropriate, isomerized to give IB

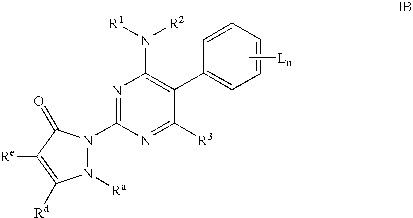

7. A process for preparing 2-substituted pyrimidines of the formula I according to claim 3 where R⁴ is a triazoldione, which comprises acylating a compound of the formula II 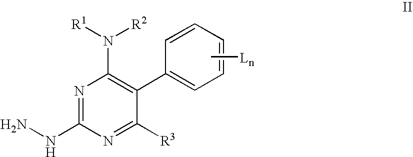
in which the substituents L, R¹, R² and R³ are with a chloroformic ester of the formula ClCO₂R where the substituent R is C₁-C₆-alkyl, giving the compound V; 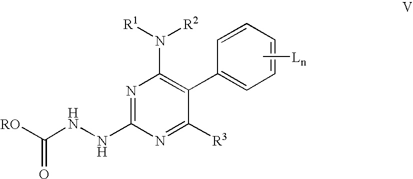
then reacting compound V with a phosgene derivative to give VI, 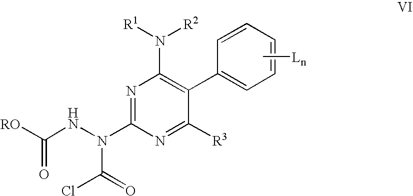
furthermore cyclizing VI with an amine of the formula R^{c}NH₂ or with a hydrazine of the formula R^{c}NH—NH₂ to give compounds ICa and Icb, respectively, and, 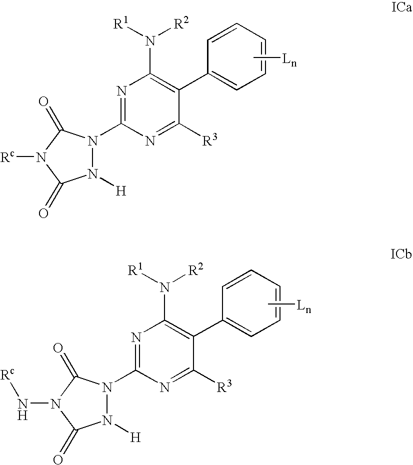
if appropriate, reacting further with an alkylating agent of the formula R^{a}X, where R^{a} is as defined above and X is a leaving group, such as halide or sulfate, to give ICa′ and ICb′, respectively.

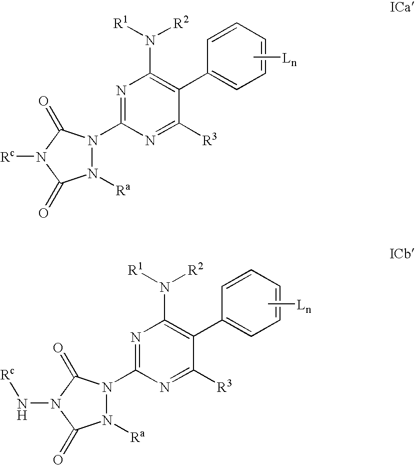

8. A process for preparing 2-substituted pyrimidines of the formula I according 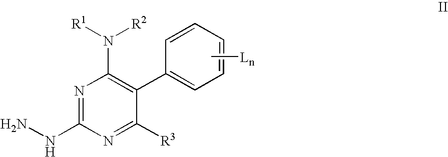
to claim 3 where R⁴ is a triazoldione, which comprises condensing a compound of the formula II in which the substituents L, R¹, R² and R³ are as defined in claim 1 with an orthoester of the formula R^{d}C(OR″)₃ where the substituent R^{d} is as defined above and R″ is C₁-C₆-alkyl, giving the compound VII; 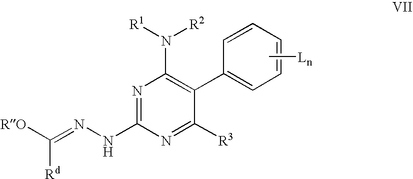
then acylating compound VII with a chloroformic ester of the formula ClCO₂R″, where the substituent R″ is C₁-C₆-alkyl, to give compound VIII 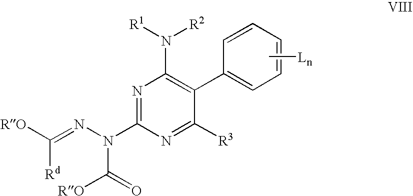
and furthermore cyclizing VIII with an amine of the formula R^{c}NH₂ to give compound ID

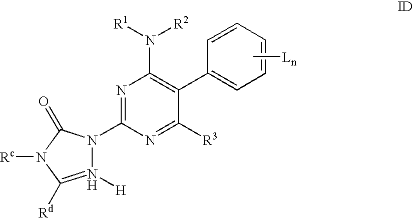

9. A compound of the formula IV 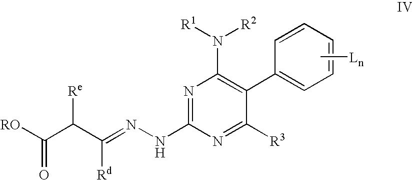
where the substituents R¹, R², R³, Lₙ, R^{e} and R^{d} are as defined in claim 1 and the substituent R is a C₁-C₆-alkyl radical.

10. A compound of the formula V 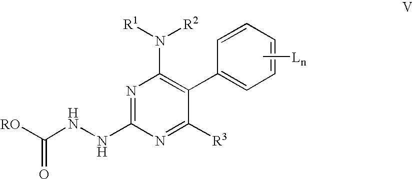
where the substituents R¹, R², R³ and Lₙ are as defined in claim 1 and the substituent R is a C₁-C₆-alkyl radical.

11. A composition suitable for controlling harmful fungi, which composition comprises a solid or liquid carrier and a compound of the formula I according to claim 1.

12. A method for controlling phytopathogenic harmful fungi which comprises treating the fungi or the materials, plants, the soil or the seeds to be protected against fungal attack with an effective amount of a compound of the formula I according to claim 1.
